(19) United States
(12) Patent Application Publication
(10) Pub. No.: US 2024/0066356 A1
(43) Pub. Date: Feb. 29, 2024

(54) Pressure-Sensing Device for Maintaining a Load-Bearing Strategy

(71) Applicants: Arick Daniel Bird, Heber City, UT (US); Derek Colon, Heber City, UT (US); Naomi Joy Cernyar, Midway, UT (US); Edwin A. Stevens, Midway, UT (US)

(72) Inventors: Arick Daniel Bird, Heber City, UT (US); Derek Colon, Heber City, UT (US); Naomi Joy Cernyar, Midway, UT (US); Edwin A. Stevens, Midway, UT (US)

(73) Assignee: Edwin A. Stevens, Midway, UT (US)

(21) Appl. No.: 17/893,823

(22) Filed: Aug. 23, 2022

(51) Int. Cl. A63B 24/00; A61B 5/00; A61B 5/103; G01L 1/18

(52) U.S. Cl. CPC A63B 24/0075; A61B 5/1036; A61B 5/6807; G01L 1/18; A61B 2562/0247; A61B 2562/043

(57) ABSTRACT

A pressure-sensing device includes an insert for insertion into a load-bearing article of clothing such as a shoe and a controller that receives analog electrical values representing an amount of load or pressure exerted on various portions of the insert. The controller incorporates an analog-to-digital converter (ADC), a processor, and a signaling unit. The ADC converts the analog electrical values into digital values at a precision associated with the ADC. If the response of the insert and ADC to load is nonlinear, a transformation function is determined that is inversely related to a curve representing that response. Application of the function to the ADC's digital outputs cancels out the nonlinearity.

### TECHNICAL FIELD

This application relates to physical therapy devices, and more particularly, to devices that help a patient implement a load bearing strategy (WBS) for conditioning or reconditioning a part of the body.

### BACKGROUND

Every year, thousands of Americans suffer from lower extremity injuries that range from ruptured Achilles, to broken Tibias, to torn ACLs. Often, the road to recovery is hard to navigate. It is a common practice for physicians to prescribe WBSs for their patients. WBSs are instructions of how much load a patient is supposed to put on the affected limb during his or her recovery period. The problem that several other researchers have discovered, and that the team that researched and developed a prototype of this application's subject matter confirm, is that patients are off by a wide margin when trying to follow their physician's directions. The team found that, on average, “patients” were off by 12% (either below or above). The team foresees that patients who ambulate continually with more than the prescribed amount of load and/or amount of time could develop several medical issues such as non-union fractures and reinjury. The team also hypothesizes that if a patient were to consistently ambulate below their prescribed load bearing strategy, they would face an increased risk of muscle atrophy and osteoporosis.

This problem has not gone unnoticed in the engineering or biotech world. Several foot sensors have been created to precisely measure the percentage of a person's load a limb is bearing at any given time. The problem is that these devices are often too expensive or too large for personal use. An example of this is the Smart-Step, a commonly used biofeedback device in this category, costing around $7,000. That is relatively cheap compared to $19,000 products that the team found. Another look at the products found that many of these biofeedback devices are large and are not able to constantly monitor a person's ability to follow a WBS. The team found that many persons wished for a mobile scale that a patient could wear while ambulating.

U.S. Pat. No. 10,470,711 to Yuan et al. describes an electric sensor system embedded in a shoe insert to provide data on the wearer's gait biometrics for use, for example, in game feedback, as well as to “construct analytical models of the wearer's gait, physical stresses, and body health.” To sense pressure, Yuan requires current to travel not only through a pressure resistive (PSR) layer but also a sensor matrix comprising a plurality of hexagonal or circular sensors. The PSR and sensor matrix are separated by an air gap. Yuan does not disclose any of the processes used to interpret the data or what kind of feedback is provided, nor does it disclose applications for maintaining a load-bearing strategy.

### SUMMARY

In one embodiment, a device is provided that assists a person in achieving a load bearing strategy for an injured portion of a leg. The device comprises a sole insert for a shoe or other article of footwear, a plurality of pressure transducers distributed across the sole insert, and a controller, including a processor, that translates or transforms readings of voltage across or current through the pressure transducers into load-bearing values and determines whether to inform or alert the patient through a signaling device. In one implementation, the plurality of pressure sensors comprise piezoresistive material, wherein the resistance across the material drops as pressure is applied to the material. In another implementation, a laminate, coating, or sheath surrounds each transducer.

The device also comprises a plurality of conductors that each connect a first end of a corresponding transducer to the controller. A neutral wire connects each of the plurality of transducers, at second ends of said transducers, to ground or neutral or an electrical return path of the circuit. The conductors carry a voltage, channel a current, or exhibit a resistance across or through the transducers that is indicative of the amount of load borne by the injured portion of the leg. In an alternative embodiment, wireless communications replace the conductors.

The controller includes a multiplexer that samples a voltage, a current, or a impedance across or through each of the transducers in order to indicate an amount of load born by the leg or an injured portion of the leg. The controller also includes one or more analog-to-digital converters that convert the sampled voltage, current, or impedance into a digital number.

If the transducers exhibit a diminished response to load, requiring progressively more additional load for each ADC output increment, the processor compensates for the diminished transducer response to load.

In one embodiment, the processor fits a nonlinear curve to a set of voltage, current, or resistance values across or through the transducers, or an average of said set of said voltage, current, or resistance values, wherein the nonlinear curve is defined by a function that converts the set of analog signals output by the ADC, or the average of said set, into a load or load value.

In another embodiment, a method is provided to implement a load bearing strategy for an injured portion of a leg. In a first step, a shoe or article of footwear is fitted with a physical therapy device. The physical therapy device comprises a sole insert, a plurality of transducers whose resistance drops when pressure is applied to the material, and a circuit that converts detected voltage, current, or impedance (e.g. resistance, reactance) values across or through the transducers into a load value. In a second step, the injured portion of the leg is inserted into the shoe or article of footwear. In a third step, load is borne on the injured portion of the leg. In a fourth step, a controller continually detects and compares an instantaneous load value with an aspirational amount of load bearing and/or a maximum allowable amount of load bearing set forth in a load-bearing strategy. In a fifth step, the controller alerts the person with the injured portion of a leg, and/or a caregiver, physical therapist, personal trainer or other person, when the instantaneous load value exceeds the recommended maximum allowable amount of load bearing.

It will be noted that not all of these steps are necessary, and it may be possible to alter the order in which these steps are performed.

In one implementation, the physical therapy device integrates or summates an amount of load borne by the injured portion of the leg over continuous time or over a sequence of small time increments. Both the integration and summation approaches fall under the umbrella of “tracking” or “accumulation.” When this cumulative amount exceeds a second threshold, the physical therapy device informs the user that it is time to rest. More complex thresholds are contemplated that are functions of both the cumulative load over both short time intervals (e.g., length of time adequate to carry out an instruction) and long time intervals (e.g., length of time adequate to complete a therapy session) as well as of the instantaneous load. For example, the physical therapy device may both inform or alert the patient when the instantaneous load exceeds a first threshold (i.e., too much load), when the cumulative load over a short time interval is below a second threshold (i.e., not enough load while carrying out an instruction), and when the cumulative load over a long time interval exceeds a third threshold (i.e., patient has done enough and can take a rest). Additional thresholds may be established for the length of time the instantaneous load exceeds a third threshold.

In one implementation, the notices and/or alerts are directed to the patient. In other implementations, the notices and/or alerts may be directed also to the caregiver, physical therapist, personal trainer or other person.

In yet another embodiment, a device is provided that assists maintenance of a load bearing strategy for an injured part of the body. The device comprises a pressure-sensitive pad, a plurality of transducers, and a processor. The pressure-sensitive pad is placed between a load and the injured part of the body. A plurality of pressure transducers is distributed across the pad, the transducers providing electrical signals that are nonlinear responses to pressure exerted on the pad. The processor translates readings across or through the pressure transducers into load-bearing values.

In one implementation, the processor uses a nonlinear function to translate the electrical signal readings into linear values indicative of an amount of load borne by the transducers. The nonlinear function may be determined by comparing a magnitude of the electrical signal to a calibrated amount of load on the pad, for each of a plurality of different loads, and fitting a nonlinear function to the measured magnitudes.

The transducers may comprise electrically conductive piezoresistive material, whose resistance drops when pressure is applied to it. Also, each transducer may be isolated from each other transducer and sandwiched between or enclosed within a second material. The transducers cross from one side of the pad to an opposite side of the pad and are spaced apart from each other.

Other systems, devices, methods, features, and advantages of the disclosed product and methods will be or become apparent to one with skill in the art upon examination of the following figures and detailed description. All such additional systems, devices, methods, features, and advantages are intended to be included within the description and to be protected by the accompanying claims.

### BRIEF DESCRIPTION OF THE DRAWINGS

The present disclosure may be better understood with reference to the following figures. Corresponding reference numerals designate corresponding parts throughout the figures, and components in the figures are not necessarily to scale.

It will be appreciated that the drawings are provided for illustrative purposes and that the invention is not limited to the illustrated embodiment. For clarity and in order to emphasize certain features, not all of the drawings depict all of the features that might be included with the depicted embodiment. The invention also encompasses embodiments that combine features illustrated in multiple different drawings; embodiments that omit, modify, or replace some of the features depicted; and embodiments that include features not illustrated in the drawings. Therefore, it should be understood that there is no restrictive one-to-one correspondence between any given embodiment of the invention and any of the drawings.

FIG. 1 illustrates one embodiment of a physical therapy device that alerts a patient when an amount of force or pressure exerted on a body part exceeds a predetermined threshold amount of force or pressure.

FIG. 2 illustrates a curve representing one embodiment of a function in which base fitting has been employed to determine a best exponential fit to data relating analog-to-digital converter outputs to actual loads.

FIG. 3 is a flow diagram that illustrates one embodiment of a process for monitoring the load borne by a leg or other load-bearing limb.

FIG. 4 is a flow diagram of one embodiment of a method for determining the load response of the physical therapy device to load borne by the leg or other load and applying a map or function to the load response values to determine the actual load.

FIG. 5 is a flow diagram of one embodiment of a method for implementing a program or plan of physical therapy while using the physical therapy device.

### DETAILED DESCRIPTION

Any reference to “invention” within this document is a reference to an embodiment of a family of inventions, with no single embodiment including features that are necessarily included in all embodiments, unless otherwise stated. Furthermore, although there may be references to “advantages” provided by some embodiments, other embodiments may not include those same advantages, or may include different advantages. Any advantages described herein are not to be construed as limiting to any of the claims.

Specific quantities (e.g., spatial dimensions) may be used explicitly or implicitly herein as examples only and are approximate values unless otherwise indicated. Discussions pertaining to specific compositions of matter, if present, are presented as examples only and do not limit the applicability of other compositions of matter, especially other compositions of matter with similar properties, unless otherwise indicated.

Where a range of values is provided, it is understood that each intervening value between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

In describing preferred and alternate embodiments of the technology described herein, various terms are employed for the sake of clarity. Technology described herein, however, is not intended to be limited to the specific terminology so selected, and it is to be understood that each specific element includes all technical equivalents that operate similarly to accomplish similar functions. Where several synonyms are presented, any one of them should be interpreted broadly and inclusively of the other synonyms, unless the context indicates that one term is a particular form of a more general term.

Any reference to “invention” within this document is a reference to an embodiment of a family of inventions, with no single embodiment including features that are necessarily included in all embodiments, unless otherwise stated. Furthermore, although there may be references to “advantages” provided by some embodiments, other embodiments may not include those same advantages, or may include different advantages. Any advantages described herein are not to be construed as limiting to any of the claims.

Specific quantities (e.g., number of mux or ADC channels and ADC precisions) may be used explicitly or implicitly herein as examples only, and other quantities are encompassed by the invention. Discussions pertaining to apparatuses and methods, if present, are presented as examples only and do not limit the applicability of other apparatuses and methods that lack certain of the exemplary components or steps and/or include additional components or steps, unless otherwise indicated.

In describing preferred and alternate embodiments of the technology described herein, specific terminology is employed for the sake of clarity. Technology described herein, however, is not intended to be limited to the specific terminology so selected, and it is to be understood that each specific element includes all technical equivalents that operate similarly to accomplish similar functions. For example, references to “load”—which has units of mass times acceleration such as “pounds”—are to be understood as including representations of mass alone, such as “kilograms.” Alternatively, references to “kilograms” may be understand as implicitly including the force of gravity.

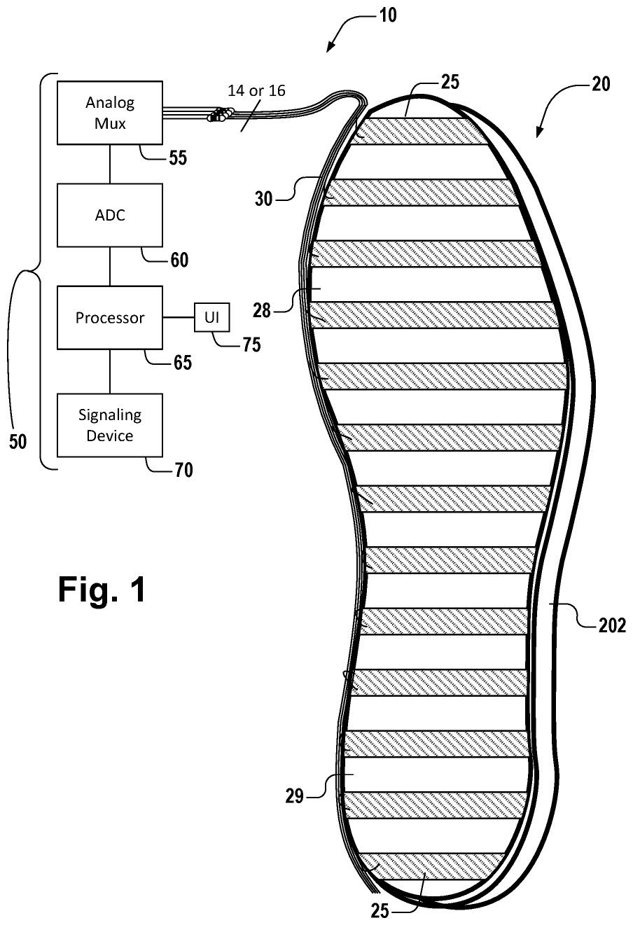

FIG. 1 illustrates one embodiment of a physical therapy device 10 that alerts a patient when an amount of force or pressure exerted on a body part exceeds a predetermined threshold. Alternatively or additionally, the physical therapy device 10 alerts a patient when the amount of force or pressure exerted is out of a range, or alternatively out of a range, of force or pressure associated with optimal healing of the body part.

The physical therapy device 10 comprises a sole insert 20, a controller 50, and cabling 30 (or a wireless medium) connecting the sole insert 20 to the controller 50. The sole insert 20 is, in different embodiments, an insole, midsole and an outsole. The sole insert 20—which in other embodiments could be substituted with a shoulder pad (for use with applicable exercise equipment), head cap (as a proxy for pressure on the neck) or glove insert (also for use with applicable exercise equipment)—incorporates a plurality of transducers 25 distributed across the sole insert 20 from at least a ball of the foot section 28 (and preferably including the area interfacing the toes) to a heel section 29.

The transducers 25 comprise flexible piezoresistive material such as Velostat, whose resistance drops when pressure is applied to the material. The transducers 25 may also comprise polymeric foil impregnated with carbon black or carbon loaded conductive polymer enclosing a first conductor that is in continuous electrical contact with the polymeric foil and extends from at least a center of the transducer to an end that protrudes outside of the enclosure formed by the polymeric foil, for connection to cable ribbon wires. The polymeric foil may be sandwiched or enclosed within a second conductor or set of conductors, which form a pressure-sensitive pad. Each transducer may further comprise a plastic or laminate covering.

The transducers 25 are arranged so that the piezoresistive material separates the first conductor from the second conductor or set of conductors. The transducers are electrically conductive and change (decrease) their resistance when pressure is applied to them.

A first end of each transducer 25 is connected to neutral or pulled to neutral via a resistor. The opposite (second) end of each transducer is electrically connected to a corresponding conductor in the cabling 30 that connects to the controller 50. In one implementation, a 1K resistor to limit the current is inserted in series between the second end of each conductor and a corresponding input of an analog mux 55 (described below). The second end of each conductor is electrically isolated from neutral by the piezoresistive material, except to the extent that current, mediated by the pressure on the piezoelectric material, allows some current to pass.

In the embodiment shown in FIG. 1, thirteen conductors of a 16-conductor cable are connected to the thirteen transducers 25 incorporated into the physical therapy device 10. A fourteenth conductor connected to the first end of each transducer 25 is connected to neutral. The two remaining conductors may be ignored, biased to high or low, or used for some other purpose.

It will be appreciated that in another embodiment, wireless communications instead of cabling 30 are used to communicate load readings to the controller 50. In one embodiment, a battery, ADC 55, and a wireless communications circuit are incorporated into the sole insert 20, eliminating the analog mux 55 and ADC 55 from the controller 50.

The controller 50 includes an analog mux 55, an analog-to-digital converter (ADC) 60, a processor 65, a signaling device 70, and a battery or other power supply (not shown). In one implementation, the analog mux 55 is digitally controlled to sample the transducers' analog signals in groups of four at a time and repeats the sampling for each group until a complete reading of all of the transducers' analog signals are obtained. The ADC 60 comprises op-amp comparators and a priority encoder, or other functionally equivalent circuitry, to convert up to four analog signal inputs into digital sensor values spanning a range of 0 to 2ⁿ−1, where n is preferably at least 8 (i.e., a precision of about 1 pound if the controller 50 is configured to measure between 0 and about 256 pounds). In one actual embodiment, n=12, and the maximum detectable load is set at 300 pounds, producing a precision of 300 divided by 4096, which is not much more than an ounce. The value of n could be as low as or lower than 5 (i.e., a resolution of 32 possible values and a precision of about 10 pounds) if rough estimates of the amount of load borne by the leg are acceptable.

The processor 65 converts the ADC output values into load values using a map and/or function that are described further below. The processor also compares the resulting load values with a threshold—the medically prescribed maximum load—to determine whether the threshold has been exceeded. If it has been exceeded, then the processor 65 causes the signaling device 70—e.g., a beeper, buzzer, strobe light, or tactile signaling device—that the patient is bearing down too much on an injured leg. The signaling device 70 could also be the patient's phone, in which case the controller 50 would include a Bluetooth communications device, or some functionally similar substitute, to communicate with the phone.

In some implementations, the controller 50 is incorporated into a pocket-sized package or device that can be kept in the patient's shirt or pants pocket, mounted on the patient's belt, worn like a wristband, or otherwise affixed to or carried by a person.

In one embodiment, it was discovered that the impedance response of the transducers was not linear throughout the range of pressures that the physical therapy device 10 encountered during use. Accordingly, efforts were made to devise a transformation function that was inversely related to a curve representing the combined response of the transducers and ADC to the load exerted on the insert 20.

There is a logarithmic function that defines a curve that partially fits empirical readings of the analog piezoelectric sensor value and the amount of pressure or force on the transducer. Likewise, there is an exponential function that transforms an equivalent portion of the empirical readings, i.e., the ADC output values, into actual load values. The exponential function is the inverse of the logarithmic function. As a practical matter, it is not necessary for the inverse function to be a mathematically true inverse. Rather, it is sufficient for the inverse function to effectively cancel out the logarithmic function. As used herein, “effectively” means that an application of the inverse function to ADC outputs into load values is, on average, accurate to within a standard deviation of 5 pounds from the actual load.

A mathematically pure exponential function fit was found to produce a very close fit for a region between 0% and about 65-80% of the maximum digital ADC output value. In the upper-value region between about 65-80% and 100% of the maximum digital ADC output value, however, the fit was poor. It appeared that the exponential relationship became more pronounced for very high digital sensor values.

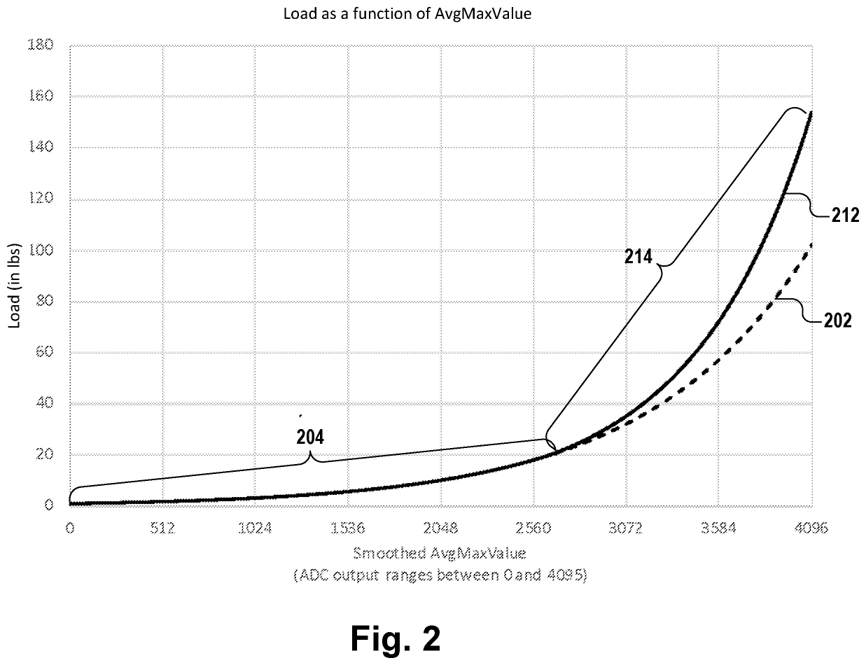

One way to deal with this would be to limit the usable range of ADC output values. For example, rather than using all 4096 values, use only values between 0 and 2700, and ignore the rest. But this is disadvantageous, because using only 2700 of the values provides less dynamic range than using all 4096 values. This is exacerbated by the fact that the upper region represents a significantly larger range of loads than the lower region, because of the logarithmic relationship between load and ADC output values. This is illustrated in FIG. 2.

Accordingly, efforts were made to identify a functionally defined adjustment to the exponential curve. Through a trial-and-error process, it was discovered that for values in the upper-value region, the base of the exponential function could be adjusted, in proportional increments, to generate a curve that closely fit the entire range of sensor-versus-actual load value pairings. FIG. 2 illustrates that curve 212.

It is convenient to show two equations (which could be combined into one or split into three or more equations) for generating this curve. The first equation finds a base and the second equation finds the power of that base raised to an ADC output or average of a group of ADC outputs:

$$base b = f ( AvgMaxValue m , threshold t ) = base {b}_{0} that fits curve below t + if ( AvgMaxVal > d ,$$

$$( m - d ) ( {( patient ' s total weight w )}^{\frac{1}{4096}} - {b}_{0} ) / ( 4096 - d ) )$$

The second equation finds the load p borne by the leg:

p=b^{m}

In the first equation, m is a dynamically changing value (e.g., as the patient walks) of between 0 and 4095. The value m is not a manually input value, but rather the dynamic output of the ADC or some combinatorially manipulated version (e.g., average) of the output (and possibly other outputs) of the ADC. In one implementation, m is AvgMaxVal, which is equal to the average of five readings for the transducers 25 centered around the transducer 25 then being read. The “max” refers to the maximum value detected during the interval the transducer 25 was read. In another implementation, AvgMaxVal is equal to the average of the maximum readings of all thirteen transducers 25.

The value d is the ADC output where the ADC response curve transitions from a fixed base exponential function to a dynamic base exponential function. The value d is predetermined; it is found through observation of the data and/or an algorithmic process for identifying a best fit of the curve defined by the equations to test data.

Finally, b is the base of the second equation. The constant b₀ is the predetermined fixed value that defines the base of the exponential function that fits the first t ADC output values. In one embodiment, b₀ is 1.00113 and d is 2650. These values require manual identification or algorithmic fitting.

The first equation could be generalized by replacing the value 4096 with 2ⁿ, where n is the bit precision on the ADC, in order to account for precisions other than the 12-bit precision ADC described herein.

It is interesting that a close fit is achieved by transitioning from a constant base to a progressively increasing base that itself is a value identified to be the best fit to the data. The use of a dynamic base to an exponential function to best-fit a data set is herein referred to as base fitting.

FIG. 2 illustrates the formulas above, where t=2650 (in units of the ADC output). The partially dashed curve 202, which overlaps the portion 204 of the best-fit curve 212 for values below t, represents the equation p=b₀^{m} for the full range of ADC output values. Applicant's base-fitting approach described above, by contrast, produces the best-fit curve 212 that has a portion 204 that rises more sharply than the corresponding portion curve 202.

The best-fit functions above are used to convert an ADC output reading (or some average thereof) into a load value that approximates and therefore represents the amount of load actually being borne by the patient's leg.

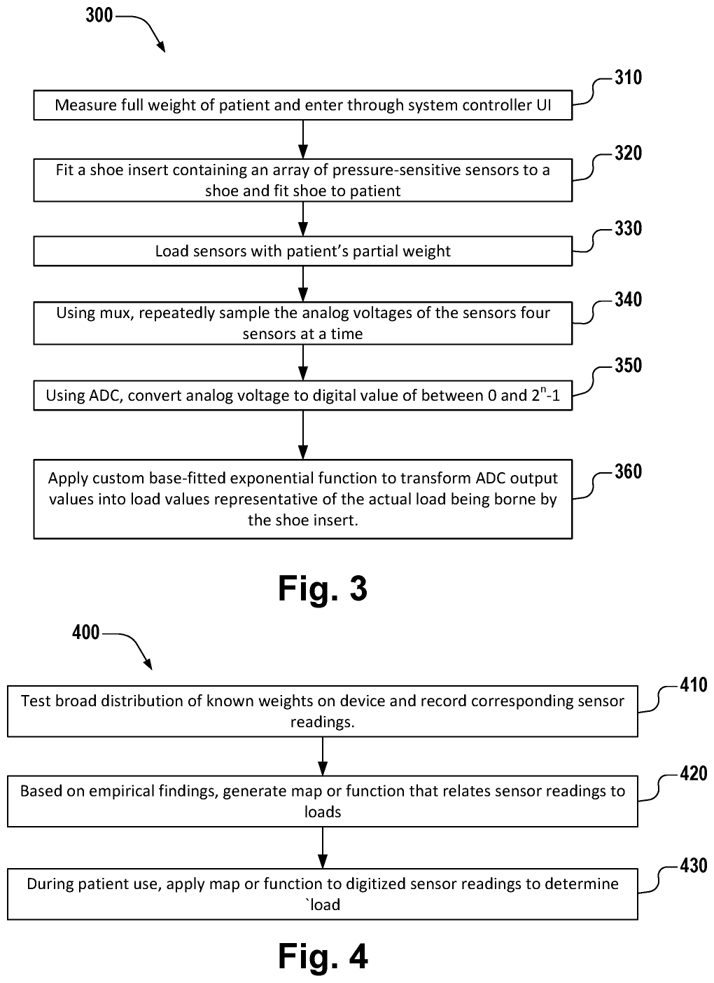

FIG. 3 is a functional flow diagram that illustrates a process 300 for monitoring the load borne by a leg or other load-bearing limb. In block 310, the patient's total load (or mass) w is measured. This information is entered into the processor through a user interface (UI) 75 associated with the processor 65. In block 320, a physical therapy device 10 is fit into a shoe and the shoe is fit to the patient's foot. The physical therapy device 10 contains an array of pressure-sensitive piezo-resistive sensors as discussed above. In block 330, the patient applies load to the the sole insert 20 by, for example, walking with the assistance of a crutch. In block 340, using the mux 55 of the physical therapy device 10, the voltages at each of the sensors are sampled four sensors at a time. In block 350, using the ADC 60, the sampled analog voltage values are converted into digital values of between 0 and 2ⁿ−1, where n is the bit precision of the ADC 60. The ADC outputs may be smoothed by averaging them with ADC outputs for adjacent transducers 25. In block 360, the custom base-fitted exponential function previously explained is applied, along with the entered patient load value and the predetermined values of b₀ and t, to the (possibly smoothed) ADC output.

FIG. 4 is a functional flow chart of one embodiment of a method 400 of calibrating the translation of ADC output values to load values. In block 410, a broad distribution of known loads is applied to the device and the ADC output recorded for each of the loads. In one implementation, each load would be distributed across the transducers 25. In another implementation, the load would be singly applied to a single transducer 25 or a combination of adjacent transducers 25, and then reapplied to the next transducer 25 or group of transducers 25, and so on in iterative fashion.

A loading machine may be used to apply a precisely calibrated load through a downwardly projecting rod. The lower end of the rod may be fitted with a tennis ball or other flexible rounded object to simulate the contact between the heel and ball of the foot and the corresponding sections of the sole insert 20. The sole insert 20 or loading machine would be iteratively forwarded or backed up to test the ADC's load response with each different transducer 15 of group of transducers 25. It will be noted that one or more groups of transducers may be defined that overlap with the preceding or following group.

In block 420, the empirical findings of block 410 are used to generate a map or function that relates the ADC output readings to the known loads. The map may be a table that relates a broadly distributed range of ADC output readings to load values, or, as discussed with FIG. 2, an equation of a curve that provides a best fit to the empirical data. There are many different techniques for fitting a curve to a paired data set, including but not limited to conventional exponential fitting, the exponential fitting enhanced with base fitting discussed with FIG. 2, logarithmic fitting, polynomial fitting, and spline fitting. The embodiment illustrated in FIG. 2 represents a transformation function that is inversely related to a curve of the combined response of the transducers and ADC to actual load borne by the patient's leg. Application of the function to the ADC's digital outputs cancels out the nonlinearity.

It is possible that the responses of the transducers 25 vary between each other. If so, separate maps or separate load response equations are generated for each transducer or each group of transducers. The processor 65 converts each actual ADC reading or group average of ADC readings into load values using the map or equation generated for the corresponding transducer 25 or group of transducers 25.

In block 430, during patient use, the map(s) or equation(s) are applied to the ADC outputs to determine the load being borne by the patient's injured leg.

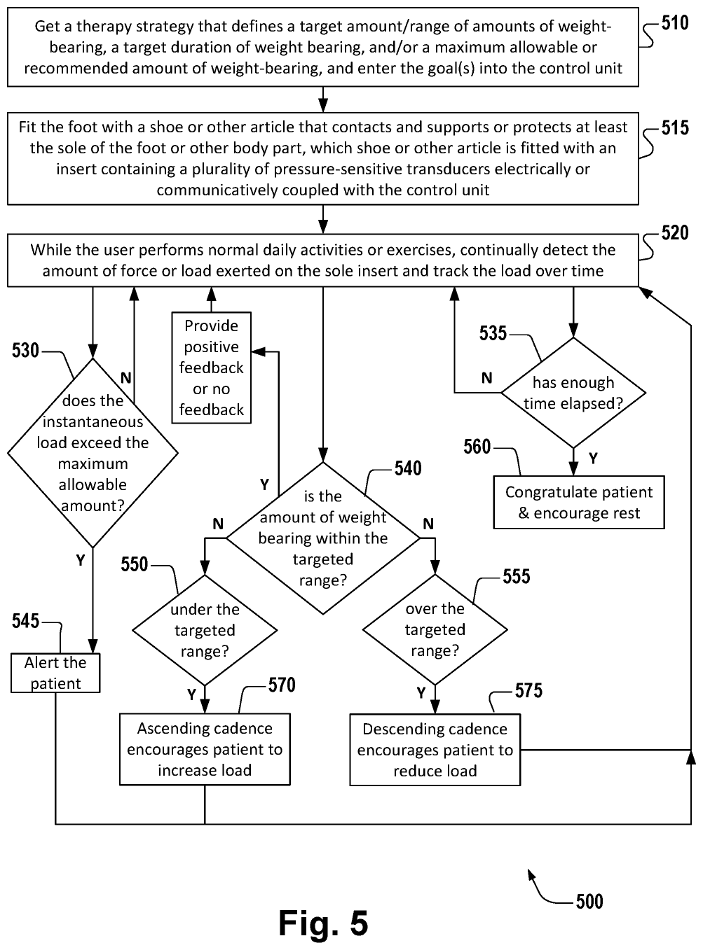

FIG. 5 is a flow diagram of one embodiment of a method 500 for assisting a program of physical therapy. In block 510, a patient gets a therapy strategy that defines a target amount or range of amounts of load-bearing during normal movements or a physical training exercise. These goals are entered into a user interface 75 of the controller 50. In block 515, an insert is fitted into a shoe or other article that contacts and supports or enshrouds at least the sole of the foot or some other body part. Alternatively, a pad is affixed to the shoulder, an insert is placed into a glove, or a similar article is affixed to some other load-bearing part of the body. The insert contains a plurality of pressure-sensitive transducers electrically or communicatively coupled with the controller 50. The shoe or other article is fit to the patient's or user's foot, limb, or other body part. In block 520, the patient or user performs their normal daily activities or prescribed exercises, while the controller 50 continually detects the amount of force or load exerted on the sole insert. The controller 50 may also cumulate and/or track the amounts over time.

In blocks 530-580, the controller 50 performs a plurality of comparisons and generates notices or alerts conditioned on those comparisons. In block 530, the controller 50 compares the instantaneous detected load with a first threshold representing the maximum recommended or allowable amount of load. In block 535, the controller 50 compares the instantaneous detected load with second and third thresholds that specify the lower and upper bounds of a target range designated to promote healing. In block 540, the controller compares the amount of time that has elapsed since the patient or user began an exercise routine with a time threshold.

Block 545 is stationed on the branch from comparison/decision block 530 that follows when the load exceeds the first threshold. In this case, the controller 50 alerts the patient. If the instantaneous load does not exceed the maximum recommended or allowable amount, flow returns to block 520.

Block 560 is stationed on the branch from block 535 indicating that the patient has completed the prescribed exercise or routine. The controller 560, possibly through a phone app, congratulates the patient or user and encourages the patient or user to take a rest. Flow proceeds from block 545 to block 520.

Blocks 550 and 555 are stationed on the branch or branches that indicate that the instantaneous detected load is lower or greater than the targeted range. If the instantaneous detected load is under the targeted range, then the controller 50 informs the patient or user and/or encourages the patient or user to increase the load on the injured leg, foot, or other body part. For example, the controller 50 may in block 570 emit or cause another electric device to emit a tonal sequence having an ascending cadence. If the instantaneous detected load is over the targeted range, then the controller 50 informs the patient or user and/or encourages the patient or user to decrease the load on the injured leg, foot, or other body part. For example, the controller 50 may in block 575 emit or cause another electric device to emit a tonal sequence having a descending cadence. Flow proceeds from blocks 545, 570, and 575 to block 520.

It will be understood that many modifications could be made to the embodiments disclosed herein without departing from the spirit of the invention.

Having thus described exemplary embodiments of the present invention, it should be noted that the disclosures contained in the drawings are exemplary only, and that various other alternatives, adaptations, and modifications may be made within the scope of the present invention. Accordingly, the present invention is not limited to the specific embodiments illustrated herein but is limited only by the following claims.

We claim:

1. A device that assists a person in achieving a load bearing strategy for an injured portion of a leg, the device comprising:
a sole insert for a shoe or other article of footwear;
a plurality of pressure transducers distributed across the sole insert; and
a controller, including a processor, that translates readings across or through the pressure transducers into load-bearing values.

2. The device of claim 1, wherein the pressure sensors comprise piezoresistive material, and the resistance across the material drops as pressure is applied to the material.

3. The device of claim 2, further comprising a laminate, coating, or sheath surrounding each transducer.

4. The device of claim 1, wherein the processor compensates for diminished transducer response to load.

5. The device of claim 1, wherein the controller includes a multiplexer that samples a voltage, a current, or a resistance across or through each of the transducers in order to indicate an amount of load born by the leg or an injured portion of the leg.

6. The device of claim 1, wherein the processor fits a nonlinear curve to a set of ADC voltage, current, or resistance output values across or through the transducers paired with actual load values applied to said transducers, and applies a function that is effectively inverse to the nonlinear curve to convert the set of ADC output values into actual load values.

7. The device of claim 6, wherein the nonlinear curve represents an exponential function, the base of which is—for at least a portion of measurable load values—a function of load value or values generated by the controller.

8. The device of claim 1, wherein the controller receives parameters of a load bearing strategy, which includes an exercise or program of exercises, and compares the parameters to real-time data of an amount load borne by the sole insert and/or an amount of time that has elapsed since the start of the program or an exercise and/or an interval of the program or exercise;
wherein at least one of the parameters is selected from a group consisting of:
a maximum recommended or allowable load on the person's leg;
upper and lower boundaries of a targeted range of loads; and
an amount of time to conduct the program or exercise.

9. A method of implementing a load bearing strategy for an injured portion of a leg, the method comprising:
inserting into a shoe or article of footwear a physical therapy device comprising a sole insert, a plurality of transducers whose resistance drops when pressure is applied to the material, and a circuit that converts detected voltage, current, or resistance values across or through the transducers into a load value;
inserting the injured portion of the leg into the shoe or article of footwear;
bearing load on the injured portion of the leg;
continually comparing an instantaneous load value detected by the physical therapy device with an aspirational amount of load bearing set forth in the load-bearing strategy.

10. The method of claim 9, further comprising informing the person with the injured portion of a leg, and/or a caregiver, physical therapist, personal trainer or other person, when the instantaneous load value detected by the physical therapy device exceeds a first threshold that is equal to or greater than the aspirational amount of load bearing.

11. The method of claim 10, further comprising:
tracking an amount of load borne by the injured portion of the leg over time;
comparing a length of time the injured portion of a leg bears at least a second threshold amount of load; and
informing the injured person, caregiver, physical therapist, personal trainer or other person when the length of time the injured portion of a leg bears at least a second threshold amount of load exceeds a third threshold amount of time.

12. The method of claim 10, further comprising:
tracking an amount of load borne by the injured portion of the leg over time;
integrating a function of at least the load borne by the injured portion of the leg above a second threshold amount of load over time; and
informing the injured person, caregiver, physical therapist, personal trainer or other person when an output of the function exceeds a fourth threshold that accounts for both the load borne by the injured portion of the leg and the amount of time said load is borne.

13. The method of claim 9, further comprises identifying a nonlinear function that fits a set of voltage, current, or impedance values across or through the transducers, or an average or weighted average of said set of said voltage, current, or resistance values, which are paired with known load values, and identifying a second function that is an effective inverse of the nonlinear function to convert another set of detected voltage, current or resistance values into measured load values.

14. The method of claim 13, wherein the second function is an exponential function, a base of which varies with at least a subset of possible voltage, current, or impedance values of the transducers.

15. A device that assists maintenance of a load bearing strategy for an injured part of the body, the device comprising:
a pressure-sensitive pad that is placed between a load and the injured part of the body;
a plurality of pressure transducers distributed across the pad, the transducers providing electrical signals that are nonlinear responses to pressure exerted on the pad; and
a processor that translates readings across or through the pressure transducers into load-bearing values.

16. The device of claim 16, wherein the processor uses a nonlinear function to translate the electrical signal readings into linear values indicative of an amount of load borne by the transducers.

17. The device of claim 16, wherein the nonlinear function is determined by comparing a magnitude of the electrical signal to a calibrated amount of load on the pad, for each of a plurality of different loads, and fitting a preliminary function to the measured magnitudes;
wherein the nonlinear function is an effective inverse of the preliminary function.

18. The device of claim 15, wherein the transducers comprise electrically conductive piezoresistive material, whose resistance drops when pressure is applied to it.

19. The device of claim 18, wherein each transducer is isolated from each other transducer and is sandwiched between or enclosed within a second material.

20. The device of claim 15, wherein the transducers cross from one side of the pad to an opposite side of the pad and are spaced apart from each other.
